(19) United States
(12) Patent Application Publication
(10) Pub. No.: US 2026/0250181 A1
(43) Pub. Date: Aug. 27, 2026

(54) GLASS CERAMIC MOLDING FOR DENTAL PURPOSES

(71) Applicant: VITA ZAHNFABRIK H. RAUTER GMBH & CO. KG, Bad Sackingen (DE)

(72) Inventors: Christopher NEUN, Bad Sackingen (DE); Michael GÖDIKER, Bad Sackingen (DE)

(21) Appl. No.: 18/878,222

(22) PCT Filed: Jul. 14, 2023
(86) PCT No.: PCT/EP2023/069626

§ 371 Date: Dec. 23, 2024

(30) Foreign Application Priority Data
Jul. 14, 2022 (EP) 22184922.7

(51) Int. Cl. C03C 4/00; A61C 13/00; A61C 13/08; C03B 32/02; C03C 3/097; C03C 10/00

(52) U.S. Cl. CPC C03C 4/0021; A61C 13/0022; A61C 13/081; C03B 32/02; C03C 3/097; C03C 10/00

(57) ABSTRACT

The present invention relates to a glass ceramic molding for dental purposes, characterized by a gradually and continuously changing weight proportion of amorphous fraction to crystalline fraction, to a method for producing same, and to its use for producing dental restorations.

The present invention relates to a glass ceramic molding for dental purposes with a gradually and continuously changing weight ratio of amorphous fraction to crystalline fraction, a process for producing it, and the use thereof for producing dental restorations.

The requirements imposed on modern tooth replacement material are numerous. Thus, the production of the individual restorations, each of which is unique, should be possible with the highest precision and a reasonable expenditure. In addition, the restoration should have a translucent optical appearance similar to that of enamel and adapt into the array of natural teeth in terms of color. In contrast, in other cases, the restoration material has to be opaque, for example, in order to be able to conceal a discolored tooth stump. Finally, the material has to withstand the chewing forces on a long-term basis, while it is simultaneously exposed to a constant attack by acids.

Glass ceramics have become established as a common material for producing dental restorations, in which mainly their strength and aesthetic properties are appreciated. The crystalline fraction not only prevents the propagation of cracks, but it also reflects and deflects light in a different way from that observed with classical glasses. A translucency that is very close to that of a natural tooth is formed thereby, which is why dental glass ceramics are employed especially in the aesthetic zone in the front tooth region.

Even though glass ceramics already have good optical properties as a tooth replacement material, the color adaptation to the natural tooth appearance remains a challenge.

EP 2 765 119 describes a blank for dental purposes which has at least 2 layers, bonded to one another, of lithium silicate glass, lithium silicate glass with nuclei or lithium metasilicate glass ceramic, wherein the layers differ in color and the layers are monolithic. Thus, it is said to be possible to well imitate optical properties of natural tooth material, and to achieve shaping without shrinkage.

WO 2013/086187 relates to a lithium silicate glass ceramic comprising 6 to 30% by weight Cs₂O, 55 to 80% by weight SiO₂, 1 to 5% by weight Al₂O₃ and B₂O₃, 7 to 16% by weight Li₂O, and 1 to 5% by weight P₂O₅, the stated weight percentages respectively being based on the total weight of the glass ceramic. In particular, blocks with a high transparency are said to be obtainable from this composition.

WO 2012/143137 A1 describes a process for preparing glass-ceramic moldings, which can be heated locally and selectively by laser treatment and induced to crystallize. Local and selectively different crystal phases can be produced selectively thereby in an amorphous glass matrix. The compositions have from 65 to 72 wt-% SiO₂, at least 10.1 wt-% Li₂O and at least 10.1 wt-% Al₂O₃, in which additionally a ratio of Li₂O to Al₂O₃ of from 1:1 to 1.5:1 is to be observed in order to form a desirable and preferred lithium aluminosilicate phase.

EP 3 974 396 A1 describes a glass ceramic comprising an amorphous phase and lithium disilicate as the dominating one. In addition, the glass ceramics contain spodumene and virgilite.

WO 2013/107653 A1 describes a process for preparing glass-ceramic moldings, which can be heated locally and selectively by laser treatment and induced to crystallize. Local and selectively different crystal phases can be produced selectively thereby in an amorphous glass matrix. The compositions have from 65 to 72% by weight SiO₂, at least 8% by weight Li₂O, and at least 8% by weight Al₂O₃, in order to form a desirable and preferred lithium aluminosilicate phase.

U.S. Pat. No. 3,464,807 describes glass ceramics having a high proportion of aluminum oxide and low proportions of lithium oxide. This results in the formation of aluminosilicates, which dominate the crystal phases. The low proportion of lithium oxide prevents lithium silicates from forming to any significant extent.

Although some efforts were made in the prior art to provide glass ceramics that come close to the aesthetic demands of dental restorations, there is still a need for improved materials, especially those which can mimic the natural color course of a tooth without perceivable transitions between the incisal, dentin and cervical regions.

In view of the above, it is the object of the present invention to provide a material for producing dental restorations, with which the optical appearance of natural tooth material can be mimicked.

This object is achieved by a molding oriented by the concept of functional graded materials (FGM), by which a material for dental restorations having a high aesthetic standard can be surprisingly achieved with at the same time comparatively low production cost.

Therefore, the present invention firstly relates to a glass ceramic molding for dental purposes comprising an amorphous fraction and a crystalline fraction, characterized in that the molding has a continuously and gradually changing weight ratio of amorphous to crystalline fractions, wherein lithium metasilicate is present as the dominating crystal phase.

Unless stated otherwise, the weight ratio of amorphous to crystalline fractions and the composition of the crystal phases can be determined by Rietveld analysis with MgO as the internal standard.

In a particularly preferred embodiment, the weight ratio of amorphous to crystalline fractions in the molding forms a gradient. Preferably, the weight ratio of amorphous to crystalline fractions is within a range of from 65:35 to 35:65, preferably from 60:40 to 40:60.

A “gradient” as used in the present invention designates the course of a gradual and continuous change of a quantity on a particular length. Said quantity may be, for example, the weight ratio of amorphous to crystalline fractions, but also physical properties.

Conventional moldings trying to mimic a natural color gradient of the tooth mostly have the disadvantage that such gradient is achieved by a layerwise build-up of the molding, wherein color transitions between the layers may have negative effects on the optical appearance. In contrast, within the scope of the present invention, it has been surprisingly found that such a color gradient can be realized also for a monolithic molding, whereby a non-visible transition in the color gradient is achieved. Therefore, an embodiment of the present invention is preferred in which said molding is monolithic.

“Monolithic” within the meaning of the present invention designates an integral one-piece molding, which is free of layers and/or interfaces.

Surprisingly, it has been found that such moldings can be used to obtain a dental restoration with a clearly more natural appearance, which can be inserted into the natural tooth scheme without complicated processing.

Without being bound by theory, it is assumed that in a gradient formed by the weight ratio of amorphous fraction to crystalline fraction, other properties of the molding also follow a gradient. In a preferred embodiment, therefore, the molding according to the invention has a gradient with respect to at least one of the properties selected from the group consisting of transmittance, Vickers hardness, especially HV3, strength, L*a*b values, and fracture toughness.

The molding according to the invention is characterized, in particular, in that it has gradients with respect to different ones of its properties, as known from “functionally graded materials”. In a preferred embodiment, the molding according to the invention is characterized in that it has no interface at a point where a gradient value of some property changes.

The molding according to the invention is characterized, in particular, by its optical properties, which are tuned for being used for dental purposes. In this context, an embodiment is preferred in which the molding has a transmittance for light of a wavelength of 360 to 740 nm of from 20 to 60%, preferably from 25 to 50%, as determined on specimens having a thickness of 1 mm.

The molding according to the invention is provided, in particular, for the production of dental restorations, and accordingly has to possess sufficient strength for processing, but also be stable enough to withstand the chewing forces. Therefore, an embodiment is preferred in which the molding according to the invention has a strength of from 200 to 600 MPa, especially from 300 to 450 MPa, as determined according to DIN EN ISO 6872 in a three-point bending experiment.

In a further preferred embodiment, the molding according to the invention has a Vickers hardness, especially HV3, of from 550 to 800, preferably from 600 to 750, as determined according to ISO EN 6507. In a particularly preferred embodiment, the molding according to the invention has a gradient with respect to its hardness. The difference in Vickers hardness in the molding according to the invention along the gradient is preferable from 10 to 100, more preferably from 20 to 80.

In order to achieve a good fit accuracy of the dental restoration, the latter can be prepared by CAD/CAM methods, which requires that the molding can be treated by subtractive processing, for example, by milling. In order to avoid crack formation and crack propagation during the processing, the molding according to the invention preferably has a fracture toughness of from 1.0 to 3.0 MPa*m^{1/2} preferably from 1.2 to 2.5 MPa*m^{1/2}, respectively determined by the SEVNB method.

The gradient of the molding according to the invention manifests itself, in particular, in color and translucency, in which these terms are mutually interchangeable. The color can be characterized, in particular, by its L*a*b* value, or by a color code usual in dental industry.

A natural tooth has a color course that follows a gradient from the gum to the cutting edge of chewing surface. In order to mimic this natural image, it has proven advantageous if the gradient follows an axis that goes through the molding. In a preferred embodiment, the gradient runs perpendicularly to the longest dimension of the molding, preferably 90° with respect to its longitudinal axis. In an alternatively preferred embodiment, the gradient runs along the longest dimension of the molding, preferably in parallel to its longitudinal axis. It has been found that this course is particularly advantageous for the machine processing of the molding with a milling and/or grinding machine.

The gradient in the molding is achieved by the continuously changing weight ratio of amorphous to crystalline fractions. In addition, an embodiment is preferred in which the crystalline fraction has different phases, in which at least two of the phases are different. In this manner, the optical properties of the molding according to the invention can be further adapted and optimized, Preferably, these phases differ with respect to at least one of the following properties:
crystal concentration;
crystal size;
crystal shape;
crystal composition;
crystal type;
crystal structure.

The mentioned parameters affect the optical properties and can therefore be used as adjusting screws for further adaptations. Thus, the appearance of the molding according to the invention can be adjusted individually by varying the parameters.

In a particularly preferred embodiment, the phases of the crystalline fraction form a gradient in the molding.

The crystalline fraction of the molding according to the invention preferably has crystals selected from the group consisting of lithium metasilicate, lithium disilicate, lithium phosphate, and mixtures thereof, in which lithium metasilicate and lithium disilicate are more preferable. The molding according to the invention has lithium metasilicate as the dominant crystal phase. “Dominant crystal phase” within the meaning of the present invention means that this crystal phase has the highest weight proportion, based on all crystal phases in the molding according to the invention. It has been found that the presence of alumosilicate crystal phases can influence both the aesthetic properties and the processability in the production of dental restorations. In a further preferred embodiment, therefore, the molding contains spodumene and/or virgilite in an amount of less than 1% by weight, more preferably less than 0.5% by weight, especially less than 0.1% by weight, and specifically less than 0.01% by weight, the indicated weights being respectively based on the total weight of the molding, and more preferably, the molding is free of spodumene and/or virgilite.

The molding according to the invention preferably contains SiO₂ and Al₂O₃, preferably in a weight ratio of SiO₂ to Al₂O₃ of from 14 to 64, more preferably from 20 to 50, and especially from 25 to 40, specifically from 25 to 35. The setting of a suitable weight ratio supports the avoidance of the formation of alumosilicates.

The molding according to the invention usually contains less than 5% by weight, preferably from 1 to 4% by weight, especially from 1.5 to 3% by weight, of Al₂O₃, the indicated weights being respectively based on the total weight of the molding.

The molding according to the invention is characterized, in particular, by its optical properties. Surprisingly, it has been found that the latter form in a particularly advantageous way if the amorphous fraction of the molding is within particular ranges. Therefore, an embodiment is preferred in which the amorphous proportion in the molding is from 30 to 70% by weight, preferably from 40 to 60% by weight, respectively based on the total weight of the molding. In this way, a translucency course corresponding to that of a natural tooth can be achieved, in particular.

In order to mimic the appearance of a natural tooth, it has proven advantageous if the amorphous proportion changes only to a low extent within the molding, wherein an amorphous minimum proportion is respectively preferred. In a preferred embodiment, therefore, the amorphous proportion in the molding changes along the gradient by at least 5% by weight, preferably by at least 7% by weight, but preferably by not more than 30% by weight, respectively based on the total volume of the molding. In a particularly preferred embodiment, the change of the amorphous proportion in the molding along the gradient is within a range of from 5 to 30% by weight, preferably from 15 to 25% by weight, respectively based on the total weight of the molding.

The properties of the molding can be achieved, inter alia, through the content of lithium disilicate and lithium metasilicate in the crystalline fraction of the molding. In a preferred embodiment, therefore, the content of lithium disilicate (Li₂Si₂O₅) in the molding changes along the gradient by at least 5% by weight, preferably at least 7% by weight, more preferably by from 5 to 20% by weight, especially by from 7 to 15% by weight, based on the total weight of the crystalline fraction.

In a further preferred embodiment, the content of lithium metasilicate (Li₂SiO₃) in the molding changes along the gradient by a maximum of 15% by weight, preferably a maximum of 10% by weight, more preferably by from 1 to 15% by weight, especially by from 3 to 10% by weight, based on the total weight of the crystalline fraction.

In a particularly preferred embodiment, the crystalline fraction has a gradient with respect to its composition. Here, the composition may change, for example, with respect to the ratio of lithium metasilicate to lithium disilicate. In a particularly preferred embodiment, the molding has a gradient of composition that ranges from a weight ratio of Li₂SiO₃/Li₂Si₂O₅ of from 50:50 to 100:0, preferably from above 50:50 to below 100:0, more preferably from above 50:50 to below 500:1, especially from 55:45 to 200:1.

The molding according to the invention is a lithium silicate ceramic. Therefore, an embodiment is preferred in which the composition of the molding contains from 50 to 75% by weight, preferably from 55 to 70% by weight, especially from 58 to 66% by weight, especially from 56 to 64% by weight, of SiO₂, especially below 60% by weight, for example, below 59.5% by weight, of SiO₂, respectively based on the total weight.

It has been found that the use of alkali oxides improves the formation of the glass phase, in which potassium oxide is advantageous, however.

The molding according to the invention preferably contains from 1 to 4% by weight, especially from 1.5 to 3% by weight, of K₂O, respectively based on the total weight of the molding.

In another embodiment of the present invention, the molding is essentially free of Na₂O and/or ZnO. Within the scope of the present invention, “essentially free” means that the proportion is below 0.5, preferably below 0.1, and specifically below 0.01, or below 0.001, % by weight, for example, 0% by weight, the indicated weights being respectively based on the total weight of the molding.

In a preferred embodiment, the molding contains P₂O₅. In one embodiment, the molding contains from 1 to 10% by weight, preferably from 3 to 8% by weight, especially from 5 to 7% by weight, of P₂O₅, the indicated weights being respectively based on the total weight of the molding.

In a further embodiment of the present invention, the molding contains zirconium dioxide. ZrO₂ has a positive effect on the strength and hardness of the moldings according to the invention. ZrO₂ is usually present in an amount of less than 15% by weight, preferably from 6 to 12% by weight, especially from 8 to 12% by weight, of ZrO₂, the indicated weights being respectively based on the total weight of the molding.

In addition, the molding according to the invention usually contains less than 6% by weight, preferably from 0 to 4% by weight, especially from 1 to 3% by weight, of CeO₂, the indicated weights being respectively based on the total weight of the molding.

The use of lanthanum oxide in the moldings according to the invention may have a positive effect of the sintering behavior. In a preferred embodiment of the molding according to the invention, La₂O₃ is contained, usually in an amount of less than 1% by weight, preferably from 0.01 to 0.5% by weight, especially from 0.05 to 0.2% by weight, the indicated weights being respectively based on the total weight of the molding.

In a particularly preferred embodiment of the present invention, the molding has the following composition:
from 56 to 64% by weight SiO₂,
from 15 to 21% by weight Li₂O,
from 1 to 4% by weight K₂O,
from 3 to 8% by weight P₂O₅,
from 1 to 4% by weight Al₂O₃,
optionally from 8 to 12% by weight ZrO₂,
optionally from 0 to 4% by weight CeO₂,
optionally La₂O₃, and
optionally from 0 to 6% by weight pigments, preferably inorganic pigments, especially metal oxide pigments,
the indicated weights being respectively based on the total weight of the molding.

In a further embodiment of the present invention, the molding comprises crystals selected from the group consisting of lithium metasilicate, lithium disilicate and lithium phosphate, and preferably, the molding contains lithium metasilicate, lithium disilicate and lithium phosphate as crystal phases with the highest proportions, and in particular, the molding essentially contains no crystal phases that have a higher weight proportion than that of lithium metasilicate, lithium disilicate or lithium phosphate.

Further, an embodiment is preferred in which the composition of the molding contains from 10 to 20% by weight, especially from 15 to 21% by weight, especially from 18 to 20% by weight, of Li₂O, based on the total weight of the molding.

Preferably, the molar ratio of Li₂O:SiO₂ in the molding is from 1.5 to 2.5, which has proven particularly advantageous for the formation of a lithium silicate glass ceramic. More preferably, the weight ratio of SiO₂ to Li₂O is from 2.6 to 4.3, especially from 2.8 to 4.1, specifically from 2.8 to 3.8, and more specifically from 2.9 to 3.4. The setting of the weight ratio promotes the formation of the lithium silicate crystal phases, especially the desired lithium metasilicate phase.

Surprisingly, it has been found that the course of the ratio of glass phase to crystalline phase and the different domains of the crystal phase as provided in the molding according to the invention can be achieved by specific thermal treatments. Therefore, the present invention further relates to a process for producing a molding according to the invention, comprising the following steps:
a) providing a glass-ceramic blank enabled by a thermal treatment to form crystals, and
b) inhomogeneous thermal treatment of the blank to obtain the molding.

The inhomogeneous thermal treatment of the blank preferably includes at least one or more inhomogeneous thermal treatments, which is the result of establishing an isothermal and/or non-isothermal heat gradient. In a particularly preferred embodiment, the process according to the invention further includes a homogeneous thermal treatment, which preferably follows the inhomogeneous thermal treatment in step b).

In order to achieve the inhomogeneous thermal treatment of the blank, preferably, a first region of the blank is treated at a temperature T₁, and a second region of the blank is treated at a temperature T₂, wherein the temperature difference between temperature T₁ and temperature T₂ is at least 5° C., preferably at least 10° C., more preferably at least 40° C., or at least 50° C., or at least 75° C. or more, and/or the temperature T₁ is preferably higher than the temperature T₂.

Here, temperature T₁ is preferably from 600° C. to 750° C., more preferably within a range of from 650° C. to 720° C., while temperature T₂ is preferably from 700° C. to 900° C., more preferably from 750° C. to 850° C.

The inhomogeneous thermal treatment of the blank is preferably effected by a contact with a heat source, wherein said contact can be direct or indirect. Said heat source may be a warming chamber, or a heatable substrate.

Preferably, the heat treatment is not effected through a laser. It has been found that a thermal treatment with a laser leads to local heating with formation of crystal nests. This can complicate the formation of a gradual and continuous course.

In a preferred embodiment, the thermal treatment is effected by inserting the blank, at least in part, preferably through a positive connection, into a warming chamber, and the warming chamber is warmed. In this way, a selective thermal treatment of individual regions of the blank can be achieved. The warming chamber is preferably a thermally conductive material having a thermal conductivity of from 50 to 500 W/(m*K), preferably from 150 to 450 W/(m*K), as determined according to heat flow calorimetry.

The thermally conductive material is preferably selected from the group consisting of non-oxide ceramics, preferably selected from Si₃N₄, silicon carbide and aluminum nitride, or metals.

The heating of the warming chamber can be adapted depending on the intensity. In a preferred embodiment, therefore, the warming chamber is heated indirectly through the ambient atmosphere, or more preferably, directly by contact with a heat source.

Alternatively and more preferably, the thermal treatment of the blank can also be achieved by a heatable substrate, on which the blank is placed. Here too, depending on the desired intensity, the contact may be indirect or direct. Therefore, an embodiment is preferred in which said heatable substrate is a heating plate onto which the blank is placed. Alternatively, the heatable substrate may also be a thermally conductive material that is heated, for example, through a heating plate, so that the heating of the blank is effected indirectly.

Also, both types of thermal treatment can be combined.

The present invention further relates to the use of the molding according to the invention for the preparation of dental restorations. Preferably, the molding according to the invention is used for producing dental restorations in the front tooth zone, especially for veneers, crowns, inlays and onlays.

The present invention further relates to a process for producing a dental restoration using the molding according to the invention, in which the process according to the invention comprises providing a molding according to the invention that has been subjected to at least a first inhomogeneous thermal treatment, and subjecting it to another thermal treatment, which is isothermal in nature. Here, the temperature of such isothermal heat treatment is preferably from 730° C. to 850° C., more preferably within a range of from 750° C. to 820° C. The holding time at this temperature is preferably from one to 15 minutes, more preferably from 3 to 10 minutes. Optionally, this process step may also be directly subsequent to the first, inhomogeneous thermal treatment in the same furnace firing. In a preferred embodiment, the process according to the invention further includes processing of the molding by machining to form the geometric shape of the dental restoration, preferably before the molding is subjected to said further heat treatment.

The present invention is described in more detail by means of the following Examples and Figures, which should by no means, however, be understood as limiting the idea of the invention.

Mixing starting components that are familiar to the skilled person followed by melting the mixture produces a blank having the following composition:

| Component | Amount in % by weight |
|---|---|
| SiO₂ | 59 |
| Li₂O | 19 |
| P₂O₅ | 6 |
| K₂O | 2 |
| Al₂O₃ | 2 |
| ZrO₂ | 10 |
| CeO₂ | 2 |
| La₂O₃ | 0.1 |
| Inorganic pigments | ad 100 |

The blank was subjected to the treatments described hereinbelow.

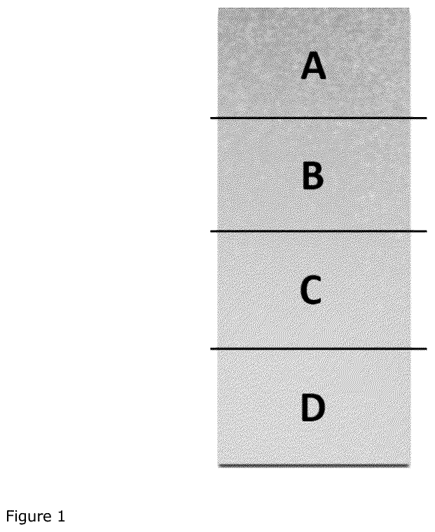

FIG. 1 shows an exemplary molding according to the invention having a gradient with respect to the ratio of amorphous fraction to crystalline fraction, and to the composition of the crystalline fraction. Table 1 shows the result of the Rietveld analysis of the molding with MgO as an internal standard, in which four equally sized sections A to D at different sites of the molding were respectively cut out and measured. The proportions are respectively stated in % by weight.

TABLE 1

| | amorphous | Li₂SiO₃ | Li₂Si₂O₅ | Li₃PO₄ |
|---|---|---|---|---|
| A | 56 | 30 | 10 | 4 |
| B | 53 | 30 | 10 | 7 |
| C | 51 | 28 | 14 | 7 |
| D | 46 | 25 | 20 | 9 |
| Δ | −10 | −5 | 10 | +5 |

Further, the transmittance and the L*a*b values were determined spectroscopically at different sites of the molding. Thus, sections having a thickness of 1 mm were cut from the molding and measured with light having a wavelength of from 360 to 740 nm. The results are summarized in Table 2:

TABLE 2

| | Transmittance | | | | |
|---|---|---|---|---|---|
| | [%] | L* | a* | b* | h* |
| 1 | 48.7 | 78.5 | 0.9 | 14.8 | 86.3 |
| 2 | 47.2 | 77.5 | 1.1 | 16.2 | 86.2 |
| 3 | 38.1 | 71.2 | 1.9 | 23.4 | 85.3 |
| 4 | 25.1 | 59.7 | 4.8 | 39.8 | 83.2 |

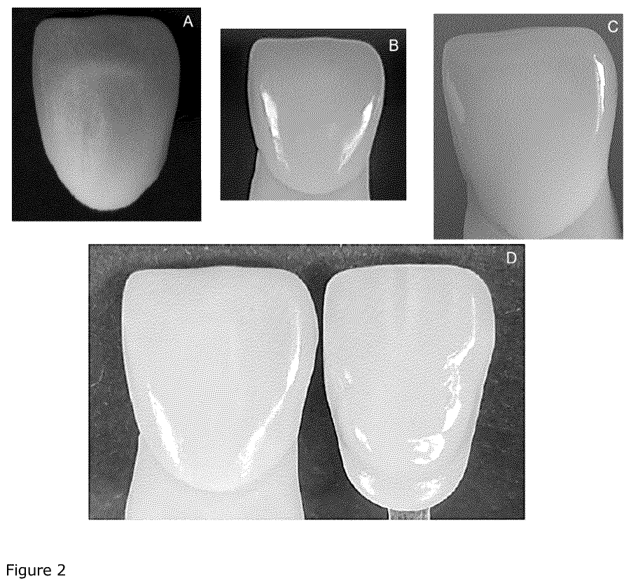

FIG. 2 shows images of a molding according to the invention, wherein

A: shows a molding according to the invention after an inhomogeneous first thermal treatment at an ambient temperature of 675° C. on a substrate heated to a temperature of 790° C.;

B: shows a molding according to the invention, which was subjected to an inhomogeneous first thermal treatment like the molding in Figure A, processed by machining to obtain the dental restoration, and subsequently subjected to a homogeneous thermal treatment at 795° C. for 8 minutes;

C: shows a molding according to the invention, which was subjected to an inhomogeneous first thermal treatment like the molding in Figure A, followed by a homogeneous thermal treatment at 795° C. for 8 minutes. Subsequently, the molding was processed by machining to obtain the dental restoration;

D: shows a molding according to the invention (left) as compared with a conventional molding according to Vita Classical Shade Guide.

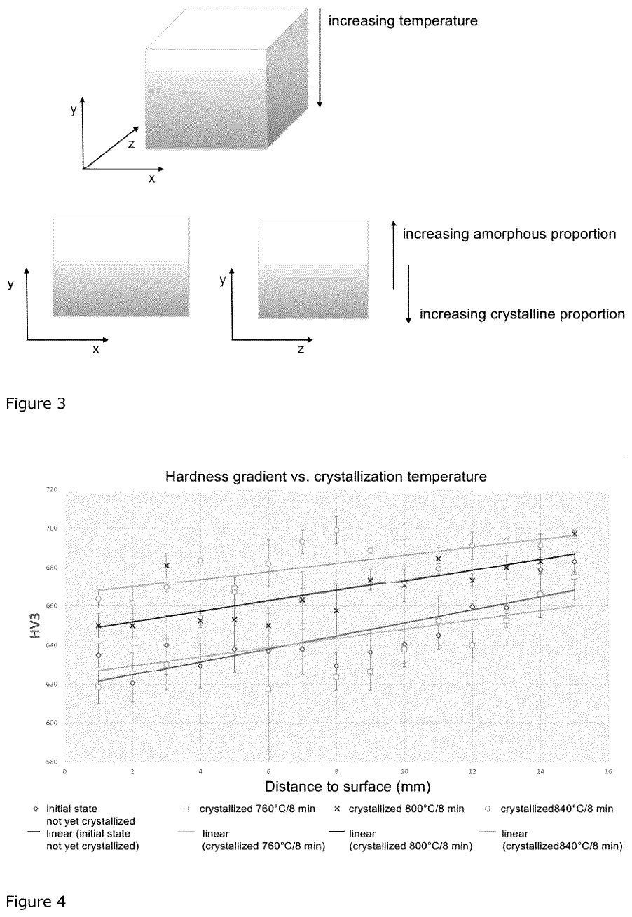

FIG. 3 schematically shows the gradual course of the ratio of amorphous to crystalline fractions in the molding as a function of the temperature course during the thermal treatment of the molding.

FIG. 4 shows a survey of the Vickers hardness (HV3) of a molding according to the invention as determined at different sites in the molding. The determination was performed in accordance with ISO EN 6507. As can be seen clearly from the survey, the molding according to the invention has a gradient with respect to the hardness according to Vickers (HV3), which also occurs during different thermal treatments.

1. A glass ceramic molding for dental purposes, comprising an amorphous fraction and a crystalline fraction, characterized in that the molding has a continuously and gradually changing weight ratio of amorphous to crystalline fractions, wherein lithium metasilicate is present as the dominating crystal phase.

2. The molding according to claim 1, characterized in that the molding contains SiO₂ and Al₂O₃ in a weight ratio of SiO₂ to Al₂O₃ of from 14 to 64.

3. The molding according to claim 1, characterized in that the molding contains spodumene and/or virgilite in an amount of less than 1% by weight, wherein the indicated weights being respectively based on the total weight of the molding and the molding is free of spodumene and/or virgilite.

4. The molding according to claim 1, characterized in that the molar ratio of Li₂O:SiO₂ in the molding is from 1.5 to 2.5.

5. The molding according to claim 1, characterized in that the weight ratio of SiO₂ to Li₂O is from 2.6 to 4.3.

6. The molding according to claim 1, characterized in that the molding contains from 50 to 75% by weight based on the total weight.

7. The molding according to claim 1, characterized in that the molding contains from 10 to 20% by weight of Li₂O based on the total weight of the molding.

8. (canceled)

9. (canceled)

10. The molding according to claim 1, characterized in that the molding contains less than 5% by weight of Al₂O₃ based on the total weight of the molding.

11. (canceled)

12. (canceled)

13. (canceled)

14. The molding according to claim 1, characterized in that the molding contains from 56 to 64% by weight SiO₂,
from 15 to 21% by weight Li₂O,
from 1 to 4% by weight K₂O,
from 3 to 8% by weight P₂O₅,
from 1 to 4% by weight Al₂O₃,
optionally from 8 to 12% by weight ZrO₂,
optionally from 0 to 4% by weight CeO₂,
optionally La₂O₃, and
optionally containing from 0 to 6% by weight pigments,
wherein the indicated weights being respectively based on the total weight of the molding.

15. The molding according to claim 1, characterized in that the molding comprises crystals selected from the group consisting of lithium metasilicate, lithium disilicate and lithium phosphate wherein the molding contains lithium metasilicate, lithium disilicate and lithium phosphate as crystal phases with the highest proportions and the molding essentially contains no crystal phases that have a higher weight proportion than that of lithium metasilicate, lithium disilicate or lithium phosphate.

16. (canceled)

17. The molding according to claim 1, characterized in that the weight ratio of amorphous fraction to crystalline fraction in the molding forms a gradient.

18. The molding according to claim 1, characterized in that said molding is monolithic.

19. The molding according to claim 1, characterized in that said molding has a gradient with respect to at least one of the properties selected from the group consisting of transmittance, strength, Vickers hardness, L*a*b values, and fracture toughness.

20. (canceled)

21. (canceled)

22. (canceled)

23. The molding according to claim 1, characterized in that the amorphous proportion in the molding is from 30 to 70% by weight based on the total weight of the molding.

24. The molding according to claim 1, characterized in that the amorphous proportion in the molding changes along the gradient by at least 5% by weight based on the total weight of the molding.

25. A process for preparing a molding according to claim 1, comprising the following steps:
a) providing a glass-ceramic blank enabled by a thermal treatment to form crystals, and
b) inhomogeneous thermal treatment of the blank to obtain the molding.

26. The process according to claim 25, characterized in that a first region of the blank is treated at a temperature T₁, and a second region of the blank is treated at a temperature T₂, wherein the temperature difference between temperature T₁ and temperature T₂ is at least 5° C. and/or wherein the temperature T₂ is higher than the temperature T₁.

27. The process according to claim 26, characterized in that the temperature T₁ is from 600° C. to 750° C., and/or the temperature T₂ is from 700° C. to 900° C.

28. The process according to claim 25, characterized in that the thermal treatment is effected by inserting the blank, at least in part, into a warming chamber, and the warming chamber is warmed, and/or the thermal treatment is effected by a direct or indirect contact of the blank with a heat source, wherein said heat source is a heating plate.

29. (canceled)

30. A process for producing a dental restoration, providing a molding according to claim 1, which has been subjected to at least one inhomogeneous thermal treatment and subjected to another thermal treatment, wherein the another thermal treatment is isothermal in nature.
